(19) United States
(12) Patent Application Publication
(10) Pub. No.: US 2023/0314397 A1
(43) Pub. Date: Oct. 5, 2023

(54) FAUCET WITH LEAD DETECT DEVICE

(71) Applicant: AS America, Inc., Piscataway, NJ (US)

(72) Inventor: Xiaojing YE, Edison, NJ (US)

(73) Assignee: AS America, Inc., Piscataway, NJ (US)

(21) Appl. No.: 18/189,064

(22) Filed: Mar. 23, 2023

Related U.S. Application Data
(60) Provisional application No. 63/326,688, filed on Apr. 1, 2022.

(51) Int. Cl. G01N 33/18; E03C 1/04; G01N 1/14

(52) U.S. Cl. CPC G01N 33/1813; E03C 1/04; G01N 1/14

(57) ABSTRACT

Provided are faucet assemblies with lead detection and reporting capabilities. In some embodiments, a faucet assembly can include a faucet body configured to be mounted above a deck; and a lead detection assembly comprising a controller, a user input device configured to receive a user input, a lead detection device, and an indicator, wherein the controller, the user input device, the lead detection device, and the indicator are in electrical communication, the user input device is configured to communicate the user input to the controller, and, based on the user input, the controller is configured to instruct the lead detection device to obtain a water sample and determine a lead content of the water sample, the controller is configured to transmit the lead content to the indicator, and indicator is configured to indicate the lead content of the water sample.

### CROSS-REFERENCE TO RELATED APPLICATIONS

This application claims the priority of U.S. Provisional Application No. 63/326,688, filed Apr. 1, 2022, the entire contents of which is incorporated herein by reference.

### FIELD

The present disclosure generally relates to faucet assemblies, and more particularly, to faucet assemblies having lead detection capabilities.

### BACKGROUND

Older plumbing fixtures and piping can include lead, if only trace amounts. When these lead-containing fixtures and piping corrode, the lead separates from the fixture and/or piping, washes away into the water running through the fixture/piping, and out of the faucet. Thus, lead-containing fixtures and piping can easily cause water to contain lead. However, lead exposure can cause any of numerous health conditions, including anemia, weakness, and kidney or brain damage. High amounts of lead exposure can even cause death.

### SUMMARY

Provided herein are faucets having lead detecting capabilities. Specifically, the faucets described herein include a lead detection device that is configured to test water during normal operation, and indicate to a user whether the lead content of the water is within or outside of predetermined limits. In some embodiments, the faucet assembly may automatically test the water as it passes through from a water source and out of the faucet. In some embodiments, the faucet assembly may manually test the water upon receiving a user input. Faucet assemblies described herein may, in some embodiments, remind to a user after a predetermined amount of time to check the lead content in the water. For example, the faucet assembly may prompt a user to provide an input to the faucet assembly initiating a lead test. In some embodiments, the faucet assembly may include an indicator that communicates to a user when the lead detection device needs to be serviced (e.g., when the lead test strip supply is running low and/or expired).

In some embodiments, the faucet assembly includes a power supply to provide power to the lead detection device and/or an indicator (e.g., LED, light). The lead detection device of the faucet assembly provides a signal to the indicator, such that the indicator can indicate any of several different messages to the user. For example, the indicator may communicate to the user that a predetermined amount of time has passed (in which case it's time for the user to initiate a lead test). The indicator may also indicate to the user that the tested lead content in the water passing through the water faucet assembly is within or outside of a predetermined limit. In some embodiments, the lead detection device may be in wireless communication with the indicator. In some embodiments, the lead detection device may be in wired communication with the indicator. The indicator may be located on the faucet assembly, for example, on the faucet body, in a conspicuous location on the faucet body.

In some embodiments, provided is a faucet assembly with lead detection and reporting capabilities, the faucet assembly comprising: a faucet body configured to be mounted above a deck; and a lead detection assembly comprising a controller, a user input device configured to receive a user input, a lead detection device, and an indicator, wherein the controller, the user input device, the lead detection device, and the indicator are in electrical communication, the user input device is configured to communicate the user input to the controller, and, based on the user input, the controller is configured to instruct the lead detection device to obtain a water sample and determine a lead content of the water sample, the controller is configured to transmit the lead content to the indicator, and indicator is configured to indicate the lead content of the water sample.

In some embodiments of the faucet assembly, the lead detection device tests the lead content using lead paper test strips.

In some embodiments of the faucet assembly, the lead detection device comprises a motor configured to transfer a lead paper test strip from a lead paper test strip supply, to a testing position for testing, and after testing a water sample, to a waste bin.

In some embodiments of the faucet assembly, the lead detection device comprises lead test paper in a roll-to-roll configuration.

In some embodiments of the faucet assembly, the lead detection device comprises a motor configured to advance the roll-to-roll lead test paper in between lead content tests.

In some embodiments of the faucet assembly, the faucet assembly comprises a power source configured to provide power to the lead detection device.

In some embodiments of the faucet assembly, the faucet assembly comprises a micropump fluidly connected to the water inlet supply and configured to obtain a water sample and pump the water sample to the lead detection device.

In some embodiments of the faucet assembly, the faucet assembly comprises a micropump located within the faucet body, fluidly connected to a water line between a mixing valve and a faucet outlet and configured to obtain a water sample and pump the water sample to the lead detection device.

In some embodiments of the faucet assembly, the lead detection device comprises a printed circuit board configured to generate a first signal representing a result of a lead content test, and send the first signal to the indicator.

In some embodiments of the faucet assembly, the indicator comprises an LED configured to illuminate a first color when the first signal represents an acceptable lead content, and a second color when the first signal represents an unacceptable lead content.

In some embodiments of the faucet assembly, the lead detection device comprises a clock.

In some embodiments of the faucet assembly, the lead detection device is configured to automatically test a water sample for lead according to a first predetermined time interval measured by the clock.

In some embodiments of the faucet assembly, the lead detection device is configured to generate and send a second signal to the indicator, wherein the second signal is based on a second predetermined time interval measured by the clock, causing the LED of the indicator to illuminate a third color.

In some embodiments of the faucet assembly, the controller and the lead detection device are configured to be positioned below the deck.

In some embodiments of the faucet assembly, the controller and the lead detection device are configured to be positioned above the deck.

In some embodiments of the faucet assembly, the indicator is positioned on the faucet body.

In some embodiments, a method of testing water for lead content is provided, the method comprising: receiving a user input at a user input device of a faucet body mounted above a deck; communicating the user input to a controller in electrical communication with the user input device; instructing a lead detection device to obtain a water sample and determine a lead content of the water sample based on the user input; transmitting the lead content to an indicator; and indicating the lead content of the water sample to a user.

In some embodiments of the method, determine a lead content of the water sample based on the user input comprises testing the lead content using lead paper test strips.

In some embodiments of the method, the lead detection device comprises a motor configured to transfer a lead paper test strip from a lead paper test strip supply, to a testing position for testing, and after testing a water sample, to a waste bin.

In some embodiments of the method, determine a lead content of the water sample based on the user input comprises testing the lead content using lead test paper in a roll-to-roll configuration.

In some embodiments of the method, the lead detection device comprises a motor configured to advance the roll-to-roll lead test paper in between lead content tests.

In some embodiments of the method, instructing a lead detection device to obtain a water sample comprises obtaining a sample of water from a water inlet supply using a micropump fluidly connected to the water inlet supply and configured to pump the water sample to the lead detection device.

In some embodiments of the method, instructing a lead detection device to obtain a water sample comprises obtaining a sample of water from a water line between a mixing valve and a faucet outlet using a micropump fluidly connected to the water line and configured to pump the water sample to the lead detection device.

In some embodiments of the method, generating a first signal representing the lead content test and transmitting the first signal to the indicator.

In some embodiments of the method, indicating the lead content of the water sample to a user comprises illuminating a first color when the first signal represents an acceptable lead content and a second color when the first signal represents an unacceptable lead content.

In some embodiments of the method, the lead detection device comprises a clock.

In some embodiments of the method, the method comprises automatically testing a water sample for lead according to a first predetermined time interval measured by the clock.

In some embodiments of the method, the method comprises generating and transmitting a second signal to the indicator, wherein the second signal is based on a second predetermined time interval measured by the clock, causing the indicator to illuminate a third color.

In some embodiments of the method, the controller and the lead detection device are positioned below the deck.

In some embodiments of the method, the controller and the lead detection device are positioned above the deck.

In some embodiments of the method, the indicator is positioned on the faucet body.

In some embodiments, any one or more of the features, characteristics, or elements discussed above with respect to any of the embodiments may be incorporated into any of the other embodiments mentioned above or described elsewhere herein.

### BRIEF DESCRIPTION OF THE FIGURES

FIG. 1 shows a front view of a faucet assembly having a lead detection device, according to some embodiments; and

FIG. 2 shows a top view of a faucet assembly having a lead detection device, according to some embodiments.

### DETAILED DESCRIPTION

Described herein are faucet assembly with lead detection capabilities. As described above, older pipes and plumbing fixtures can contain lead that, when the pipes and/or plumbing fixtures corrode, end up in the water flowing through the pipes. Once lead gets in the water flowing through the piping and plumbing fixtures, it can get into human bodies. Lead exposure can cause any of numerous health conditions, including anemia, weakness, kidney damage, brain damage, and even death.

Accordingly, the faucet assemblies provided herein provide lead detection capabilities with a lead detection device. A faucet assembly having lead detection capabilities can alert a user to an unsafe or unacceptable lead content in their water. Early detection can allow a user to act sooner, minimizing the total amount of lead exposure and the risk of suffering from any of the above health conditions associated with lead exposure.

The lead detection device can be located below deck (i.e., below the countertop within which the sink is installed). In some embodiments, a lead detection device and a controller of the lead detection device may be located below deck. In some embodiments, a lead detection device and a controller of the lead detection device may be located above deck (e.g., in or adjacent a faucet body). The lead detection device of the faucet assembly can automatically test the water for lead, and/or the lead detection device can test the water for lead in response to a user input. The faucet assembly can include a user indicator that can, upon receiving a signal from the lead detection system, send a message to the user. In some embodiments, the indicator can illuminate a specific color to indicate a specific message (e.g., green (“first color”) for acceptable lead content, red (“second color”) for unacceptable lead content, yellow (“third color”) to remind user to test the lead content, etc.). In some embodiments, the indicator may include a plurality of lights, wherein each light is configured to illuminate a different color, each color representing a different message or status.

Referring now to the drawings, like parts are marked throughout the specification and drawings with the same reference numerals, respectively.

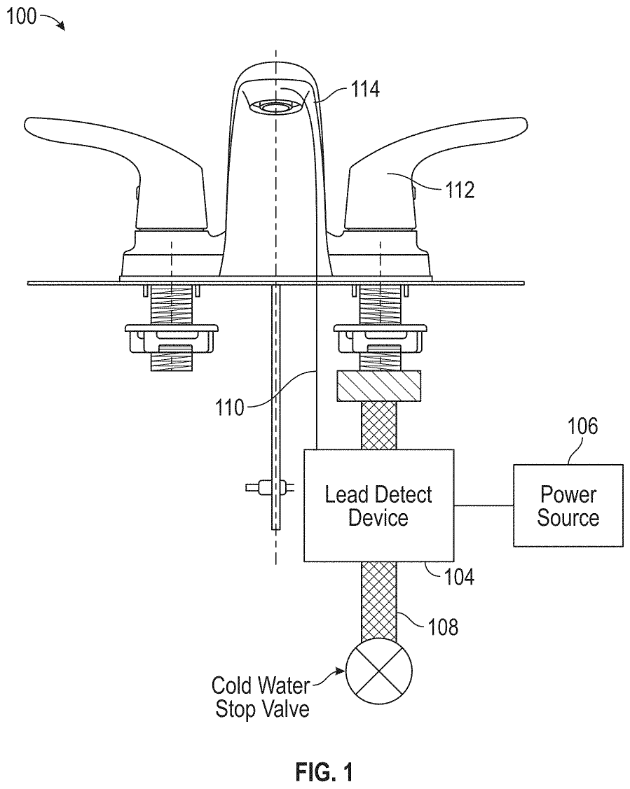

FIG. 1 shows a front view of a faucet assembly 100 having a lead detection device 104, according to some embodiments. As shown, the faucet assembly 100 can include a faucet body 114, one or more handles 112, lead detection device 104, power source 106, cold water supply line 108, and wired connection 110.

In some embodiments, the lead detection device 104 may be in-line with the cold water supply line 108. As water flows from a cold water supply to the faucet, it passes thorough the lead detection device 104 via cold water supply line 108. Power source 106 is configured to supply power to lead detection device 104 and/or an indicator. Power source 106 can include AC power, DC power, or a combination of AC and DC power.

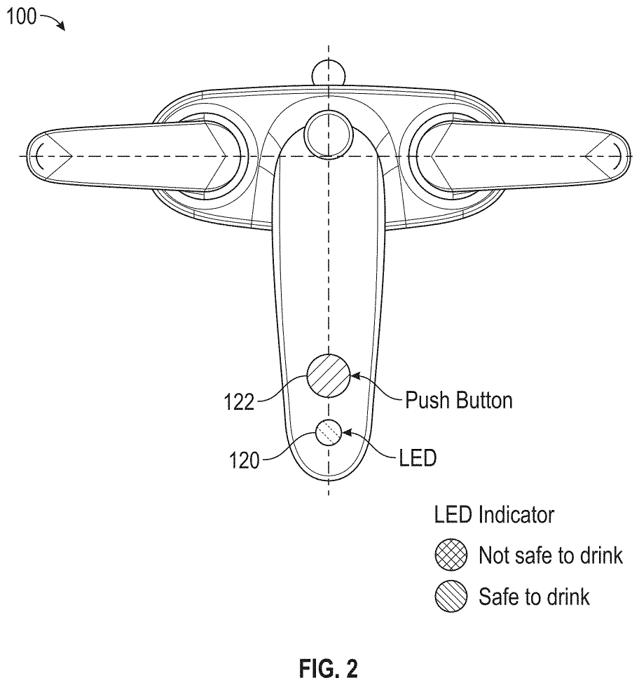

In some embodiments, the power source 106 is always on. For example, in embodiments that include a clock integrated into a controller (e.g., PCB) of the lead detection device 104, for example, the power source 106 must always be on to power the clock and automatically run a lead test at the predetermined timed intervals. In some embodiments, the faucet assembly 100 includes a push button (e.g., user input device 122 of FIG. 2) that is configured to turn the power source 106 on, which then automatically runs a lead test.

Lead detection device 104 can utilize lead test paper to test the water as it passes through the cold water supply line 108. Lead test paper changes color when in contact with water to indicate the amount of lead in the water. The lead test paper strips are one-time use, meaning that once a test strip has been used to test a water sample, it must be discarded. Accordingly, in embodiments utilizing lead paper test strips to test the water, the lead test paper must be supplied to the lead detection device 104 and replaced once the supply of lead paper test strips is exhausted. Further, in some embodiments, the lead detection device 104 comprises a motor configured to advance a test strip from a lead paper test strip supply to a testing position. The water sample can be pulled from the main cold water supply line 108 and directed to a positioned lead paper test strip (for example, via a micropump). Once the water contacts the lead paper test strip and the lead paper test strip is allowed time to react to the water, the used test strip is directed away from the testing position. In some embodiments, the used test strip is directed into a waste bin. The waste bin can collect used test strips, and be empty on occasion by a user. In some embodiments, the lead detection device 104 includes a motor and/or conveyor system to advance the lead paper test strip from a supply, to the testing position, and/or to the waste bin.

In some embodiments, a visual indicator may communicate to a user when the lead paper test strip supply needs to be replenished. For example, the lead paper test strip supply corresponding to the lead detection device 104 may include a sensor (e.g., level sensor, presence sensor, etc.) that is configured to determine when the lead paper test strip supply is low or completely empty. The sensor can be configured to send a signal to an indicator to display (e.g., illuminate) a signal to the user indicating that the lead paper test strip supply is low/empty. In some embodiments, the lead paper tests strips may be provided as a cartridge of a plurality of individual test strips. In some embodiments, the lead paper may be provided as a continuous roll-to-roll strip, that is configured to advance after each test, to provide a new, unused portion of the lead test paper in a testing position (i.e., where the water sample is inserted into the lead detection device 104).

In some embodiments, the faucet assembly may include a micropump. The micropump can remove a small sample of water from the cold water supply line 108, and pump the water sample to lead detection device 104 to be tested.

In some embodiments, lead detection device 104 may be provided as a retrofit kit to implement into an existing faucet. In that case, the indicator communicatively coupled to the lead detection device 104 may be detached from the faucet body.

In some embodiments, lead detection device 104 is provided under a deck (e.g., countertop) or water receptacle (e.g., sink) as shown in FIG. 1. However, in some embodiments, the lead detection device 104 is provided within the faucet body 114. For example, lead detection device 104 may be provided between a mixing valve and the faucet outlet. In some embodiments, just a micropump of a lead detection device 104 may be located within the faucet body 114. For example, the micropump may be located between a mixing valve and a faucet outlet of faucet body 100. The micropump may take a sample of water from a water line directing mixed water from the mixing vale to the faucet outlet, and carry the sample to a testing position of lead detection device 100 (which may be located under the deck and/or receptacle).

FIG. 2 shows a top view of a faucet assembly 100 having a lead detection device, according to some embodiments. As shown, FIG. 2 shows an indicator 120 and a user input device 122 positioned at a top surface of the faucet body. However, indicator 120 may also be located elsewhere, such as, for example, on a deck. In some embodiments, a “lead detection assembly” may include a controller, a user input device 122, a lead detection device, and an indicator 120.

Although the indicator 120 and the user input device 122 are both positioned at a top surface of the faucet body of faucet assembly 100 proximate to the spout, they could be located in other places as well. For example, indicator 120 and user input device 122 may be located near a base of the faucet body or on an escutcheon of faucet body assembly 100. Further, although FIG. 2 shows a physical push button as user input device 122, note that user input device 122 may be any suitable user input device such as, but not limited to, a pressure sensor, a capacitive sensor, a switch, etc.

In some embodiments, faucet assembly 100 may comprise user input device 122, which can allow a user to manually initiate a lead test of the water. Upon receiving a user input, the user input device 122 may send a signal to lead detection device 104, causing lead detection device to initiate a lead test. In some embodiments, the user input may be sent from user input device 122 to a controller, and the controller may instruct the lead detection device 104 to obtain and test a water sample for lead content, based on the user input. In some embodiments, lead detection device 104 may include a clock, such that a lead test may automatically be initiated at predetermined intervals (e.g., daily, weekly, monthly, bimonthly, etc.). In some embodiments, a faucet assembly 100 may include both manual and automatic testing capabilities.

Upon testing the water for lead, a controller (e.g., printed circuit board (PCB)) of the lead detection device may be configured to convert the result of the lead test into an electrical signal that can be sent to indicator 120. For example, if the lead paper test strip changes to a color indicating a lead content within a predetermined acceptable limit, a controller of the lead detection device can generate a first signal. The first signal is configured to be set to indicator 120 and cause indicator 120 to illuminate a color corresponding to an acceptable lead level (e.g., green). If the lead paper test strip changes to a color indicating a lead content outside a predetermined acceptable limit, a controller of the lead detection device can generate a second signal. The second signal can be sent to indicator 120 and cause indicator 120 to illuminate a color corresponding to an unacceptable lead level (e.g., red). In FIG. 2, a red indicator color is conceptually illustrated using a first hashing pattern and a green indicator color is conceptually illustrated using a second hashing pattern. The controller can be in electrical communication with the user input device 122, the lead detection device 104, and/or the indicator 120 to allow the signals to be transmitted between the various components.

In some embodiments, the faucet assembly 100 must be turned off (i.e., water should not be actively passing from the water source, through the faucet assembly, and out of the faucet outlet). The faucet should be turned off in some embodiments to enable the lead detection device to more easily obtain a sample (i.e., it can be easier to obtain a sample of water to test when the water is still and not flowing). In some embodiments, if a user initiates a lead test while the faucet is running, the result of the lead test may not be accurate. In some embodiments, the faucet assembly includes a mechanism by which user input device 122 is not able to be pushed when the faucet is running. For example, in some embodiments, the faucet assembly may comprise a flow sensor. When the flow sensor senses water flowing through the faucet assembly, it can send a signal to a controller (e.g., PCB) of the lead detection device such that a lead test is prevented from running. In some embodiments, the mechanism may include a switch associated with one or more faucet handles. For example, a switch might be triggered by turning a handle, which would break a circuit. This circuit break could be communicated to the controller, which would not allow any measurement. In some embodiments, breaking the circuit would itself disallow any sample taking.

Generally, the lead content in water is highest after the faucet has been sitting, unused for a period of time (e.g., overnight). In some embodiments, an instruction manual for the faucet assembly may instruct a user to turn on the faucet assembly, let it run for a certain amount of time to flush out any built-up lead content, and then test the water.

The foregoing description sets forth exemplary systems, methods, techniques, parameters, and the like. It should be recognized, however, that such description is not intended as a limitation on the scope of the present disclosure but is instead provided as a description of exemplary embodiments.

Although the description herein uses terms first, second, etc. to describe various elements, these elements should not be limited by the terms. These terms are only used to distinguish one element from another.

1. A faucet assembly with lead detection and reporting capabilities, the faucet assembly comprising:
a faucet body configured to be mounted above a deck; and
a lead detection assembly comprising a controller, a user input device configured to receive a user input, a lead detection device, and an indicator,
wherein the controller, the user input device, the lead detection device, and the indicator are in electrical communication, the user input device is configured to communicate the user input to the controller, and, based on the user input, the controller is configured to instruct the lead detection device to obtain a water sample and determine a lead content of the water sample,
the controller is configured to transmit the lead content to the indicator, and
indicator is configured to indicate the lead content of the water sample.

2. The faucet assembly of claim 1, wherein the lead detection device tests the lead content using lead paper test strips.

3. The faucet assembly of claim 2, wherein the lead detection device comprises a motor configured to transfer a lead paper test strip from a lead paper test strip supply, to a testing position for testing, and after testing a water sample, to a waste bin.

4. The faucet assembly of claim 1, wherein the lead detection device comprises lead test paper in a roll-to-roll configuration.

5. The faucet assembly of claim 4, wherein the lead detection device comprises a motor configured to advance the roll-to-roll lead test paper in between lead content tests.

6. The faucet assembly of claim 1, comprising a power source configured to provide power to the lead detection device.

7. The faucet assembly of claim 1, comprising a micropump fluidly connected to the water inlet supply and configured to obtain a water sample and pump the water sample to the lead detection device.

8. The faucet assembly of claim 1, comprising a micropump located within the faucet body, fluidly connected to a water line between a mixing valve and a faucet outlet and configured to obtain a water sample and pump the water sample to the lead detection device.

9. The faucet assembly of claim 1, wherein the lead detection device comprises a printed circuit board configured to generate a first signal representing a result of a lead content test, and send the first signal to the indicator.

10. The faucet assembly of claim 9, wherein the indicator comprises an LED configured to illuminate a first color when the first signal represents an acceptable lead content, and a second color when the first signal represents an unacceptable lead content.

11. The faucet assembly of claim 1, wherein the lead detection device comprises a clock.

12. The faucet assembly of claim 11, wherein the lead detection device is configured to automatically test a water sample for lead according to a first predetermined time interval measured by the clock.

13. The faucet assembly of claim 11, wherein the lead detection device is configured to generate and send a second signal to the indicator, wherein the second signal is based on a second predetermined time interval measured by the clock, causing the LED of the indicator to illuminate a third color.

14. The faucet assembly of claim 1, wherein the controller and the lead detection device are configured to be positioned below the deck.

15. The faucet assembly of claim 1, wherein the controller and the lead detection device are configured to be positioned above the deck.

16. The faucet assembly of claim 1, wherein the indicator is positioned on the faucet body.

17. A method of testing water for lead content, the method comprising:
receiving a user input at a user input device of a faucet body mounted above a deck;
communicating the user input to a controller in electrical communication with the user input device;
instructing a lead detection device to obtain a water sample and determine a lead content of the water sample based on the user input;
transmitting the lead content to an indicator; and
indicating the lead content of the water sample to a user.

18. The method of claim 17, wherein determine a lead content of the water sample based on the user input comprises testing the lead content using lead paper test strips.

19. The method of claim 17, wherein determine a lead content of the water sample based on the user input comprises testing the lead content using lead test paper in a roll-to-roll configuration.

20. The method of claim 17, wherein instructing a lead detection device to obtain a water sample comprises obtaining a sample of water from a water inlet supply using a micropump fluidly connected to the water inlet supply and configured to pump the water sample to the lead detection device.

21. The method of claim 17, wherein instructing a lead detection device to obtain a water sample comprises obtaining a sample of water from a water line between a mixing valve and a faucet outlet using a micropump fluidly connected to the water line and configured to pump the water sample to the lead detection device.

22. The method of claim 17, comprising generating a first signal representing the lead content test and transmitting the first signal to the indicator.
